(19) United States
(12) Patent Application Publication
(10) Pub. No.: US 2017/0097255 A1
(43) Pub. Date: Apr. 6, 2017

(54) AEROSOL MASS SENSOR AND SENSING METHOD

(71) Applicant: KONINKLIJKE PHILIPS N.V., EINDHOVEN (NL)

(72) Inventor: KORAY KARAKAYA, EINDHOVEN (NL)

(21) Appl. No.: 15/316,854

(22) PCT Filed: Jun. 10, 2015
(86) PCT No.: PCT/EP2015/062872

§ 371 Date: Dec. 7, 2016

(30) Foreign Application Priority Data
Jun. 10, 2014 (EP) 14171818.9

(51) Int. Cl. G01G 3/16; B01D 53/00; G01N 1/22; G01N 33/00; G01N 5/04; G01N 15/06

(52) U.S. Cl. CPC G01G 3/16; G01N 5/04; G01N 15/0637; G01N 1/2202; G01N 33/0062; B01D 53/005; G01N 2015/0046

(57) ABSTRACT

A mass sensor for measuring particle mass within an aerosol uses resonance frequency detection to determine a mass of particles. A heating element is used for heating the resonant sensor element and it is controlled during a sensing cycle, with the change in mass of the deposited particles monitored during heating. This enables a low cost device to be able to detect particle concentration as well as provide information about the chemical and/or physical nature of the particles.

### FIELD OF THE INVENTION

The invention relates to the measuring of particle mass within an aerosol and the identification of the types of particle present.

### BACKGROUND OF THE INVENTION

Airborne particle pollution, especially particle matter size less than 2.5 μm diameter range (named “PM2.5”), is a big concern for countries like China, where the speed of industrialization stretches the boundaries of regulatory requirements.

As a consequence of increasing consumer empowerment, the demand for information about the air quality of living spaces is increasing. Especially in China, excessive PM2.5 pollution has become a common problem in the last decade. This problem is also validated by continuous measurements in various Chinese cities. The data is publicly available and can be simultaneously monitored by mobile phone applications or through the web.

Availability of this data as well as continuous national and international media attention has created strong consumer awareness about the problem.

Official outdoor air quality standards define particle matter concentration as mass concentration per unit volume (e.g. μg/m³). The average PM2.5 pollution concentration in mainland China has been calculated based on satellite data, and it has been found that the majority of the country exceeds the World Health Organization limits of 10 μg/m³, with some regions reaching and even exceeding PM2.5 concentrations of 100 μg/m³.

Standardized reference measurement methods are based on measuring the mass of deposited or captured particles per air sampling volume for example using a quartz crystal microbalance, a tapered resonator, an impactor, or weighing filters and sieves.

However, these systems require professional operational guidelines for handling the manual part of the measurement (e.g. weighing a filter and sieve) and/or periodic maintenance for cleaning the accumulated mass, maintaining various system components and recalibration.

Mass measurement in this way also does not provide any information about the chemical and physiochemical nature of the particles themselves.

Ambient aerosols, indoor and outdoor, consist of various species with different chemical and physical properties depending on their origin. Different types include volatile/semi-volatile species (e.g. nitrates and sulphates), hydrocarbons (poly aromatic hydrocarbons), various carbon species (e.g. soot, smoke) and inorganics, bio aerosols (bacteria, viruses, pet dandruff, dust mite excretes, and fungi spores).

Analysis of the physical and chemical properties of aerosols provides additional information that can be linked to their health effects. These physical and chemical properties can also be used for identifying the origin of aerosols, and this information will enable a better estimation about of the particle size distribution, shape, density, etc.

There are methods for chemical analysis of particulate matter. For example, thermo gravimetric analysis is a well-known analytical technique that can also be applied for characterization of aerosols of unknown origin. The method involves measuring the change of the weight of an unknown sample under controlled heating conditions (e.g. controlled heating rate). As a result, it is possible to identify the type and the ratio of volatile and reactive species in a mixture of substances.

Although thermo gravimetric analyses of aerosols provide a set of useful information, it is typically performed in established laboratory settings with dedicated equipment.

There is therefore a need for a miniaturized system to enable a low cost analytical device suitable for consumer applications.

Resonance-based mass sensing for aerosol contamination monitoring has been proposed. For example, use of a micromachined silicon cantilever device with a picogram level of mass resolution for personal exposure monitoring has been proposed. Filters can be used for eliminating large particles and an electrostatic sampler can be provided for depositing nanoparticles on the cantilever. For example WO 2013/064157 discloses a MEMS based resonant particle measurement device, designed for measuring aerosol nanoparticles in an air flow stream.

However, this approach does not provide any chemical analysis.

It would be desirable to provide a low cost device which enables both particle concentration to be determined (based on mass sensing) as well as some chemical and physical information of the collected particles. Known thermo gravimetric analyses tools are designed for laboratory establishments and not suitable for use at consumer settings.

### SUMMARY OF THE INVENTION

The invention is defined by the claims.

According to an embodiment of the invention, there is provided a mass sensor for measuring particle mass within an aerosol, comprising:

a sensor element;

a heating element for heating the sensor element;

a transducer element for driving the sensor element into resonance and detecting the resonance frequency of the sensor element, wherein the resonance frequency is dependent on a mass of particles deposited on the sensor element; and

a controller for operating the heating element during a sensing cycle and monitor a change in the mass during heating based on a detected change in the resonance frequency.

The aerosol may be air or any other gas with entrained particles.

This sensor arrangement is controlled such that, after particulate material has been deposited on the sensor element (e.g. during a first phase of the sensing cycle), heating is carried out. The mass of particulate material deposited on the sensor will change during heating for example due to temperature-dependent evaporation. The way the mass varies with temperature (in particular reduces), detected based on the resonance frequency varying with temperature, can be used to obtain information about the nature of the deposited particles.

The controller may be further adapted to:

implement an initial sampling operation with no heating; and

perform a subsequent temperature control.

In this way, the sensor is initially controlled to attract a sample, following which temperature control is used to determine a function of the sample with respect to temperature.

A look up table is preferably provided comprising information relating to the mass-temperature function for different types of particulate material.

In this way, a comparison of the mass function with temperature can be made with previous stored results (which may be based on calculations or on calibration information) to enable the types of particulate material to be obtained.

The sensor element may comprise any resonance based sensor can be used which provides sufficient mass resolution, for example from picograms to milligrams. The sensor element may for example comprise a MEMS sensor. This enables a low cost and compact sensor to be fabricated.

A MEMS sensor element may for example be formed as a clamped-clamped resonator beam or a clamped-free resonator beam.

The heating element may comprise a heating track formed on the surface of the resonator body or embedded in the resonator body. This enables integration of the heating element into the structure of the sensor. The resonator body can have low thermal mass so that a low power heater is required.

Alternatively, the heat can be applied using an external heater element, for example an infrared lamp, or a resistive heater in the close vicinity of the resonator sensor.

In all cases, the heater element preferably provides a controllable heating rate (i.e. temperature slope with respect to time) throughout the testing period.

A sample intake device is preferably provided for operating during at least a first part of the sensing cycle to drive the aerosol being monitored towards the sensor element. The sensor is then only exposed to the particulate aerosol during the sensing operation, so that the lifetime is prolonged.

The sample intake device may be a fan or a pump. Alternatively, an electrostatic attraction arrangement may be provided. Further alternatives comprise gravity based deposition of particles, or thermophoretic deposition, or use of natural convection.

A particle filtration arrangement may be used for defining a range of particle sizes for which the aerosol contamination is to be analysed.

This means the behavior of particles can be monitored only for a range of particle sizes of interest. The filtration may be based on a mechanical filter or based on aerodynamic separation such as using an impactor.

The sensor may further comprise a gas sensing element in the vicinity of the sensor element, to detect the nature of the gases or vapors emitted from the sensor as the temperature increases. A gas sensor can also be used to detect the concentration change of reactive gases. For example a decrease in oxygen concentration will be indicative of consumption by an oxidation reaction, and various chemical reactions may occur at elevated temperature.

An embodiment of the invention also provides a method of measuring particle mass within an aerosol, comprising:

driving a sensor element into resonance;

detecting the resonance frequency of the sensor element, wherein the resonance frequency is dependent on a mass of particles deposited on the sensor element;

heating the sensor element; and

monitoring the change in mass during heating based on a detected change in the resonance frequency.

This method monitors changes in mass during heating of the sensor element. The characteristics of the mass-temperature function enable information concerning the nature of the deposited particles to be obtained.

An initial sampling operation can be carried out with no heating, and a subsequent temperature control can then be carried out.

In this way, the sensor is initially controlled to attract a sample, following which temperature control is used to determine a function of the same with respect to temperature.

The invention also provides an air treatment device, comprising a mass sensor of the invention.

### BRIEF DESCRIPTION OF THE DRAWINGS

Examples of the invention will now be described in detail with reference to the accompanying drawings, in which:

FIG. 1 shows the fundamental aspects of a resonance based mass detection explained with a spring mass system, where the mass of the resonator influences the resonance frequency;

FIG. 2 shows the basics of thermo gravimetric information;

FIG. 3 shows an embodiment of the sensor of the invention.

FIG. 4 shows an embodiment of the method of the invention; and

FIG. 5 shows an embodiment of the resonator element with integrated heater used in the system and method of the invention.

### DETAILED DESCRIPTION OF EMBODIMENTS

The invention provides a mass sensor for measuring particle mass within an aerosol, in which resonance frequency detection is used to determine a mass of particles. A heating element is used for heating the resonant sensor element and it is controlled during a sensing cycle with the change in mass of the deposited particles monitored during heating. This enables a low cost device to be able to detect particle concentration as well as provide information about the chemical and/or physical nature of the particles.

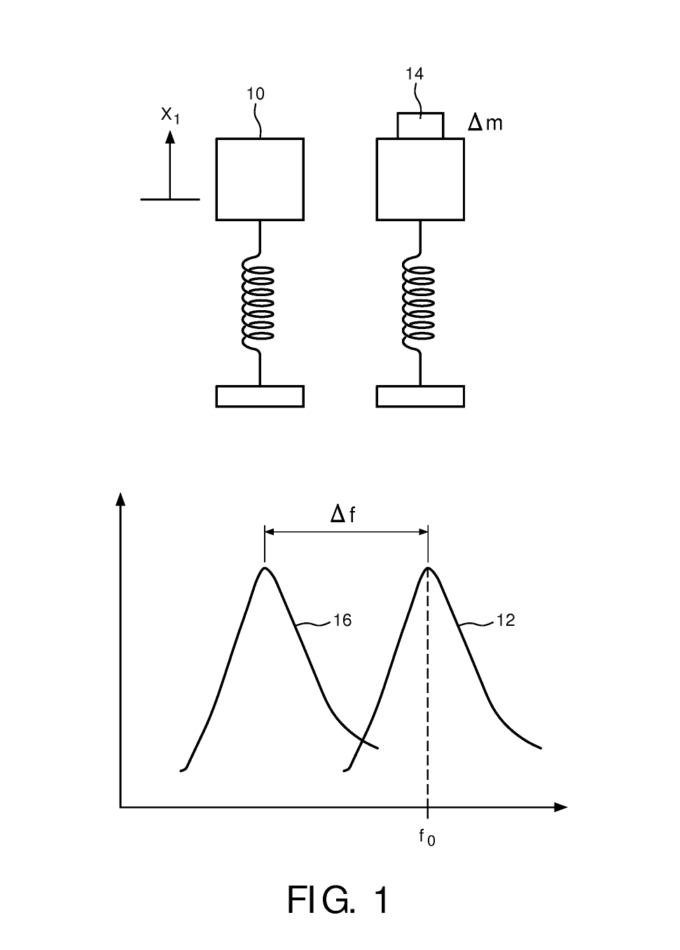

Direct mass measurement using resonant devices is a known technique. It is based on well-known principles based on the relationship between the resonance frequency (f₀) and the mass of a resonator, as shown in FIG. 1.

In FIG. 1, a resonator mass 10 is represented schematically, with a mass m and spring constant k. The graph shows the amplitude of the resonant oscillations (on the y-axis) as a function of frequency (the x-axis). Plot 12 is for the basic resonator mass. If an additional mass 14 is added (Δm) the oscillation curve shifts down in frequency to plot 16 with a frequency shift Δf.

The equations which govern the resonant vibrations are;

$$\begin{matrix}{f}_{0} = \frac{1}{2 π} \sqrt{\frac{k}{m}} & ( 1 ) \\ Δ f = - \frac{1}{2} \frac{Δ m}{m} {f}_{0} & ( 2 ) \\ Δ {m}_{min} ∝ \frac{m}{Q} & ( 3 )\end{matrix}$$

Equation 1 shows the relationship between the basic resonance frequency and the resonator characteristics. Equation 2 shows the change in frequency caused by a change in mass, and equation 3 shows the minimum mass (Δmₘᵢₙ) that can be detected. The minimum depends on the mechanical quality factor Q of the resonator.

There are several examples of resonance based mass sensing for aerosol contamination monitoring in literature. For example, use of a micromachined silicon cantilever device with a picogram level of mass resolution for personal exposure monitoring has been proposed. Filters can be used for eliminating large particles and an electrostatic sampler can be provided for depositing nanoparticles on the cantilever.

For example WO 2013/064157 discloses a MEMS based resonant particle measurement device, designed for measuring aerosol nanoparticles in an air flow stream.

In the same way that the resonance frequency drops with increased deposited mass, the resonance frequency increases if the mass decreases over the resonator, e.g. by means of evaporation.

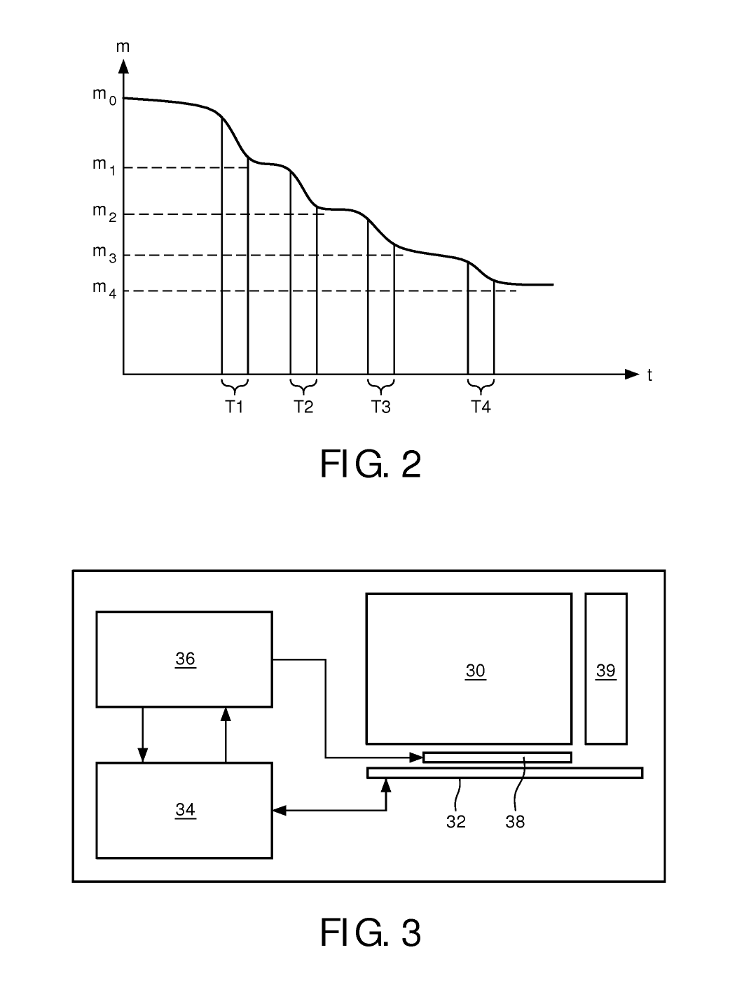

The basics of thermogravimetric information are shown in FIG. 2, for a hypothetical aerosol deposit. The graph shows the change in mass with respect to temperature. The change in mass can be measured by the mass sensor over time, and if there is a known temperature profile with respect to time, the curve of FIG. 2 can be obtained.

Different stages of the weight change profile (weight loss in this case) correspond to certain events, which can be associated with the presence of a certain type of aerosol.

For example, the drop in mass during temperature range T1 may correspond to moisture loss. The drop in mass during temperature range T2 may correspond to evaporation of a first semi-volatile compound. The drop in mass during temperature range T3 may correspond to evaporation of a second semi-volatile compound. The drop in mass during temperature range T4 may correspond to burning and gasification of remaining organic aerosols. Obviously, some reactions, such as oxidation in solid state, can result an increase in the measured mass and in this case the thermogravimetric profile will indicate an upwards shift at the temperature range corresponding to that reaction.

The concentration (weight percent) of different compounds in an aerosol mixture can be calculated by subtracting the corresponding mass values (m₁, m₂, etc.) form the original mass (m₀) of the aerosol deposit.

The thermal properties of aerosols of various origins have been investigated in the literature. The article of Perrino, Cinzia, et al. “Thermal stability of inorganic and organic compounds in atmospheric particulate matter.”, in Atmospheric Environment 54 (2012): 36-43 discloses an investigation of the thermal behaviour of atmospheric particulate matter. It discloses thermogravimetric analysis used to detect mass losses.

The article of Wittmaack, Klaus, and Lothar Keck, “Thermodesorption of aerosol matter on multiple filters of different materials for a more detailed evaluation of sampling artifacts.”, in Atmospheric Environment 38.31 (2004): 5205-5215 also discloses gravimetric mass analysis of samples after step-wise thermodesorption.

The temperature range of thermal degradation/evaporation of various aerosols is therefore known.

The invention may for example make use of these information sources either in the form of a built-in or online look up table. It is also possible to obtain the relevant information by experiment, for example using samples of materials which are intended to be detected by the mass sensor.

In a preferred solution, it is possible to update a look up table by software upgrade or by using an online database for the data analysis part of the proposed sensor.

The invention is based on the use of a heating element for heating the resonant sensor element so that a change in mass of the deposited particles on the resonant sensor element can be monitored during heating, based on a detected change in the resonance frequency.

The detailed design of the sensor will depend on the application conditions.

In general, as shown in FIG. 3, the sensor system comprises a particle pre-classification unit 30 and intake sampling device (e.g. a filter stack), a MEMS resonator 32 (described below), an electronic circuit 34 for driving and reading out the sensors and other system components and a controller 36 for data processing and storage. Air flow to the sensor unit can be handled by using fans and/or thermal convection.

The MEMS resonator 32 incorporates a heater element 38 which is controlled by the controller 36 to perform a heating cycle.

The sample intake and conditioning unit 30 is designed taking into account the targeted particle range. A specific particulate matter range (e.g. PM1, PM2.5, PM10) may be targeted by using appropriate particle size pre-classification methods; e.g. a mesh/filter combination or inertial separation. Providing enough sample air volume, consistently over the service lifetime is the key parameter for designing such a system. Particle filters such as fibrous filters, meshes, inertial and aerodynamic separation units may be used for particle size range selection.

Deposition of the particles can be controlled by electrostatic or electrophoretic precipitation of charged particles on a grounded or oppositely biased resonator. Thermophoretic precipitation may instead be used which comprises creating a temperature difference between the resonator and a counter surface. The deposition may instead be based on random particle movement.

A fan, a pump, or a convection unit for delivering the sampled air volume may also be used to design the system to be compatible with this key parameter.

The selection depends on the minimum detectable mass, average particle concentration in ‘clean air’ (baseline level), ratio of particles passing through the particle filters in sampling subsystem and eventually the user requirements for minimum particle concentration detection.

A MEMS resonator may be used as the resonant sensor element 32. The resonator can be designed and fabricated with suitable dimensions to achieve the desired resonance frequency for providing the required limit of detection.

Examples of possible resonator structures are cantilevered structures (one end clamped, other end free), as well as double-clamped or membrane type resonators.

A cantilevered design may be of particular interest for providing sufficient electric field density at the cantilever tip in the case of electrostatic particle collection. A cantilevered structure can be in simple rectangular form, in triangular form (for a larger clamping area) or in hammerhead-like form for increasing the surface area while maintaining the low area at the clamped end.

These parameters all affect the resonance behavior of the system and basic resonator design principles can be used.

The circuitry 34 for driving and reading out the resonance frequency also depends on the Q-value of the resonator, choice of transducer (e.g. piezoelectric, thermal, piezoresistive, optical, capacitive, etc.). Depending on the requirements for minimum detectable mass, a Q-compensation mechanism may be implemented for increasing the mass resolution of the system. The detection of the resonance frequency in the electronic domain is selected to be suitable for the actuation method. Fundamentals of circuit design for such resonators are known in literature.

For example, in the case of piezoelectric actuation and sensing, an oscillator circuit is used which incorporated the electrical impedance of the resonator. In the case of electrostatic/capacitive actuation and sensing a voltage controlled oscillator circuit is used.

The controller 36 for data processing and handling can also be selected and designed depending on the application requirements such as the data sampling rate, processing load for calculations and implementation of data processing algorithms. The controller interfaces with the electronic circuit 34 as well as providing control of the heater element 38.

The sensor may further comprise a gas sensing element 39 in the vicinity of the sensor element 32, to detect the nature of the gases or vapors emitted from the sensor as the temperature increases. A gas sensor can also be used to detect the concentration change of reactive gases. For example a decrease in oxygen concentration will be indicative of consumption by an oxidation reaction, and various chemical reactions may occur at elevated temperature. A gas sensor provides

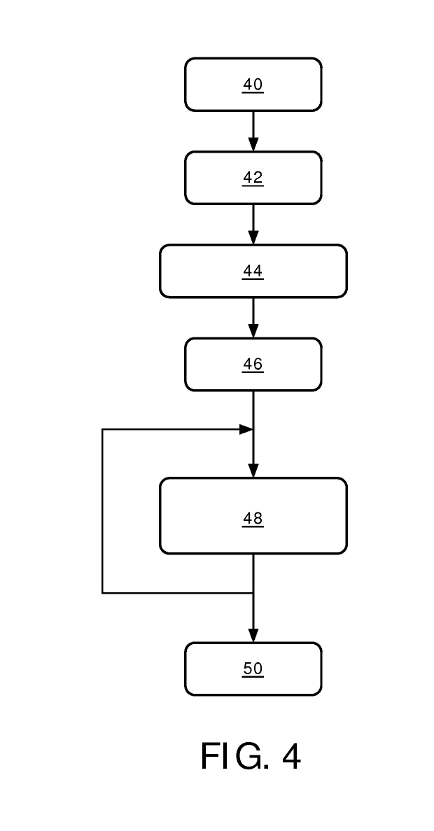

FIG. 4 shows the method of using the sensor.

In step 40, the initial resonance frequency (f₀) is measured (i.e. at time t₀).

The cycle starts in step 42, for example by starting an air intake (for example with a fan, at a known air flow rate).

After a fixed volume of air has been sampled, the resonance frequency is measured in step 44 and a change in mass m₀ is obtained.

In step 46, the heater is activated. During heating, the resonance frequency is monitored in step 48 until the heating cycle is finished. Monitoring the resonance frequency is used to track changes in the resonance frequency Δf.

The recorded frequency profile with respect to time is processed to derive a mass profile with respect to time. This is converted to a mass versus temperature profile, and from the mass versus temperature response, chemical and/or physical information can be derived. This processing all takes place in step 50. The function of temperature with respect to time can be obtained based on the known response of the resonator element to the heating power provided to it, or else there may be temperature sensing and feedback to assist in preparing a temperature versus time profile. This temperature versus time profile is used to convert the mass versus time profile to a mass versus temperature profile.

The processing step 50 may comprise comparing data from the thermo gravimetric profile (of FIG. 2) with a look-up table that contains information relating to compounds originated from different indoor aerosol generating events. This information is then used for identification of the aerosol generating event and the expected particle size distribution for that particular type of event.

This information can then for example be used for optimizing an air filtration process. For example, an event which generates aerosols with high moisture content is typical for cooking activities and/or bio aerosols, which can be discriminated by the additional information about volatile compound ratios.

The mass sensor provides an output which thus indicates the concentration of particles of a particular size range and also gives information about the nature of the particles. This information may be used to control an air treatment device. For example, a high recorded mass indicating high levels of pollution can give rise to a high capacity mode of operation (e.g. by selecting a high fan speed setting for an air purifier device), and a low recorded mass can give rise to a lower capacity mode of operation. In this way, energy savings are obtained, and the lifetime of the air purifier device can be prolonged.

Different air treatment devices may be activated depending on the detected type of particulate pollution, so that the air treatment process can be tailored to the type of pollution that it present.

In the case of an air treatment device which controls the air properties of a space by using air intake from outdoors, the sensor reading can be used for regulating the air intake from outdoors, for example depending on whether or not the outdoor air pollution exceeds the system requirements as well as depending on the type of pollution detected.

The sensor readings (or concentration and type of particle) may be provided to the user as an output, for example using a display screen. The user can then process and respond to the information accordingly, and the sensor can be a stand alone sensor device. Alternatively, the sensor readings may function as an internal control parameter within a larger system, which reacts automatically in response to detected levels of pollution. This larger system may be an air purifier or other air quality control system.

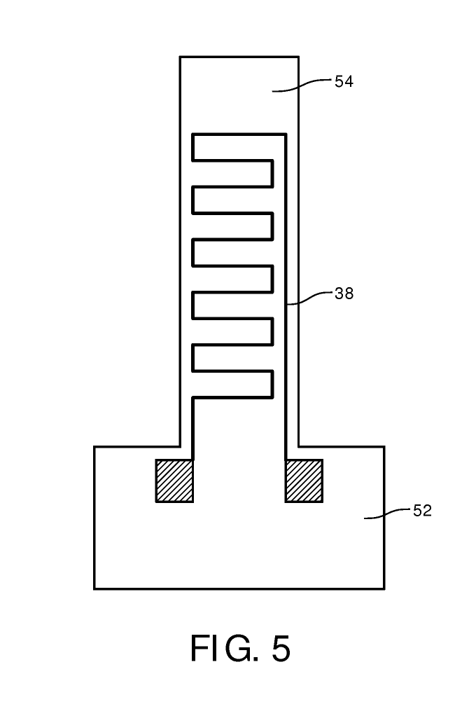

In one example the heater 38 is formed on the resonator surface, for manipulating the temperature in a controlled manner. A schematic representation of a micro resonator is given in FIG. 5. A metal (or other conductor) wire, with a known resistance can be used for structuring the heater 38 on the resonator surface. The resonator shown in FIG. 5 has a cantilever design, with a fixed end 52 which is anchored to a substrate, and a free end 54.

The metal can be chosen according to the desired temperature range. For example, for elevated temperatures suitable for monitoring combustion processes (e.g. burning of organics in normal air ambient for detecting the organic content of aerosols), materials should be selected suitable for temperatures in the range 700° C.-800° C. For example, a combination of silicon micro resonator and TiN heater, which are typical materials used in MEMS manufacturing processes, is suitable for this purpose.

Using a micro resonator system also enables low power operation, since the thermal mass of the system is very small compared to bulk systems, hence does not need large power consumption for heating. Low thermal mass and integrated heater wires also enable strict control of the resonator temperature by using the known linear relationship between the heater wire resistance and the temperature (i.e. negative temperature coefficient of resistance). Thus, resistance measurement provides a mechanism for providing temperature feedback, using the heating element itself, instead of requiring a separate temperature sensor.

Thermal insulation may be provided between the resonator and the anchor/substrate by providing an opening which can be implemented by bulk micromachining techniques (e.g. deep reactive ion etching), as a part of the MEMS manufacturing process of the resonator (e.g. during the resonator release step). An opening for providing thermal insulation may preferably be achieved by back-side etching of the wafer, in which or which the resonator is built. In the case of a silicon (or other semiconductor) wafer, one preferred method would be to use a silicon-on-insulator type wafer, and the thermal insulation properties of the insulator layer can then be used for preventing excessive thermal energy loss to the bulk of the substrate material.

The example above makes use of a heater element which forms an integral part of the sensor. However, the heat can be applied using an external heater element, for example an infrared lamp, or a resistive heater in the close vicinity of the resonator sensor. A combination of heating elements can be used.

The examples above are based on detection of PM2.5 particles, but the invention can be applied to PM10, PM1 particles or other categories of ultrafine particles.

The example above is based on a MEMS resonator. However, the approach can be based on other micro resonators, for example a membrane device (similar to a capacitive micromachined ultrasound transducer) or a quartz crystal microbalance (QCM). The resonator may be a bulk acoustic wave (BAW) resonator, or a surface acoustic wave resonator (SAW).

The invention is applicable to air purifiers, stand-alone particle sensor units, personal exposure monitoring devices, vehicle cabin particle measurement sensors, particle sensors for outdoor use (as a standalone sensor unit or for example, sensors for lamp posts for city management), ventilation units, various parts of a building climate management system and in general various types of mass sensors. There are also medical applications in respiratory support and drug delivery applications.

The system makes use of a controller. Components that may be employed for the controller include, but are not limited to, conventional microprocessors, application specific integrated circuits (ASICs), and field-programmable gate arrays (FPGAs).

In various implementations, a processor or controller may be associated with one or more storage media such as volatile and non-volatile computer memory such as RAM, PROM, EPROM, and EEPROM. The storage media may be encoded with one or more programs that, when executed on one or more processors and/or controllers, perform at the required functions. Various storage media may be fixed within a processor or controller or may be transportable, such that the one or more programs stored thereon can be loaded into a processor or controller.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. An air treatment device comprising:
a mass sensor for measuring particle mass within an aerosol, the mass sensor comprising:
a sensor element;
a heating element for heating the sensor element;
a transducer element for driving the sensor element into resonance and detecting the resonance frequency of the sensor element, wherein the resonance frequency is dependent on a mass of particles deposited on the sensor element; and
a controller for operating the heating element and monitor a change in the mass during heating based on a detected change in the resonance frequency,
wherein the controller is further adapted to,
compare the change in mass information with information stored in a look-up table, thereby identifying aerosol generating events and expected particle size distributions for the identified events; and
control the air treatment device in dependence on the identified aerosol genertaing events and corresponding particle size distributions.

2. A device as claimed in claim 1, wherein the controller is further adapted to:
implement an initial sampling operation with no heating; and
perform a subsequent temperature control.

3. A device as claimed in claim 2, further comprising the look up table, which comprise information relating to the mass-temperature function for different types of particulate material.

4. A device as claimed in claim 1, wherein the sensor element comprises a MEMS sensor having a resonator body.

5. A device as claimed in claim 4, wherein the heating element comprises a heating track formed on the surface of the resonator body or embedded in the resonator body.

6. A device as claimed in claim 1, further comprising a sample intake device for operating during at least a first part of a sensing cycle to drive the aerosol being monitored towards the sensor element.

7. A device as claimed in claim 1, further comprising a particle filtration arrangement for selecting a range of particle sizes for which the particle mass is to be measured.

8. A device as claimed in claim 1, further comprising a gas sensing element in the vicinity of the sensor element.

9. (canceled)

10. A method of controlling an air treatment device comprising:
measuring a particle mass within an aerosol, the measuring comprising:
driving a sensor element into resonance;
detecting the resonance frequency of the sensor element, wherein the resonance frequency is dependent on a mass of particles deposited on the sensor element;
heating the sensor element; and
monitoring the change in the mass during heating based on a detected change in the resonance frequency,
comparing the change in mass information with information stored in a look-up table, thereby identifying aerosol generating events and expected particle size distributions for the identified events; and
controlling the air treatment device in dependence on the identified aerosol genertaing events and corresponding particle size distributions.

11. A method as claimed in claim 10, comprising:
implementing an initial sampling operation with no heating; and
performing a subsequent temperature control.

12. A method as claimed in claim 10, wherein the look up table comprises information relating to the mass-temperature function for different types of particulate material to obtain particle information from the monitored change in mass during heating.

13. A method as claimed in claim 10, wherein the heating element comprises a heating track formed on the surface of the resonator body.

14. A method as claimed in claim 10, further comprising driving the aerosol being monitored towards the sensor element during at least a first part of a sensing cycle.

15. A method as claimed in claim 10, further comprising performing particle filtering for defining a range of particle sizes for which the aerosol contamination is to be monitored.
